SEQUENCE LISTING (6 sequences)

SEQ ID NO: 1 - DNA, 22 bp, Homo sapiens
tgtcttgtgaaacaaaatgctt

SEQ ID NO: 2 - DNA, 21 bp, Homo sapiens
aaaagtctgctggtctgccta

SEQ ID NO: 3 - DNA, 27 bp, Homo sapiens
aatgctttttaacatccatataaagct

SEQ ID NO: 4 - DNA, 25 bp, Homo sapiens
ccttaatttaaggaatgtgagcacc

SEQ ID NO: 5 - DNA, 16 bp, Artificial
  artificial MGB probe
cagggtttcagacaaa

SEQ ID NO: 6 - DNA, 18 bp, Artificial
  artificial MGB probe
tgattttgtctaaaaccc

(19) United States
(12) Patent Application Publication
(10) Pub. No.: US 2006/0088873 A1
(43) Pub. Date: Apr. 27, 2006

(54) Methods for SMN genes and spinal muscular atrophy carriers screening

(75) Inventor: Yi-Ning Su, Taipei City (TW)

Correspondence Address: BACON & THOMAS, PLLC, 625 SLATERS LANE, FOURTH FLOOR, ALEXANDRIA, VA, 22314, US

(73) Assignee: Yi-Ning Su, Taipei City (TW)

(21) Appl. No.: 11/256,943

(22) Filed: Oct. 25, 2005

(30) Foreign Application Priority Data
Oct. 26, 2004 (TW) 093132418

(51) Int. Cl. C12Q 1/68; C12P 19/34

(52) U.S. Cl. 435/6; 435/91.2

(57) ABSTRACT

A method for SMN genes identifying is disclosed, as well as a method for spinal muscular atrophy carriers screening. The method comprises steps of following: (a) providing a genomic DNA; (b) amplifying the genomic DNA with a pair of primers; and (c) injecting the amplified product into DHPLC (Denaturing High Performance Liquid Chromatography). The method of the present invention can identify SMA patients, and also the carriers of SMA.

### BACKGROUND OF THE INVENTION

1. Field of the Invention

The present invention relates to a genomic detecting method and, more particularly, to a detecting method for SMN genes and Spinal muscular atrophy carriers.

2. Description of Related Art

Motor neuron disease (MND) is one kind of neurodegenerative disease, and one of the MNDs is spinal muscular atrophy (SMA). SMA occurs due to the mutation of survival motor neuron gene (SMN) on chromosome 5 which causes degeneration of the motor neurons of the spinal cord anterior horn cells and results in muscular atrophy. Normally the muscles start to atrophy from the palms, interphalangeal muscles, shoulders, neck, tongue, and swallowing and breathing muscles which ultimately lead to deaths of dysphagia and respiratory failure. In Taiwan there is an overall incidence of 1 in 10000 live births and a carrier percentage of 1%-3%.

Somatic chromosomes come in pairs, which means there are two sets of genes on each chromosome. If a set of genes on somatic chromosomes mutates, e.g. deletion, the other set is still able to produce sufficient functional proteins; therefore, this kind of genomic abnormality will not manifest clinical symptoms which constitute inherited recessive disorders, e.g. SMA. If the carriers are normal in phenotype, the genes are heterozygote. If parental genotypes are both heterozygote, the progeny will receive a set of inherited recessive genes respectively; therefore, the progeny with SMA will have the characteristics of the inherited recessive disorder, and the genotype of the progeny is homozygote.

Normally there are two almost identical copies of the SMN genes on chromosome 5 in a human body, including the SMN1 near the telomere, and the SMN2 near the centromere. Only a few normal people have SMN1 but not SMN2. These two SMN genes have only five base pairs difference in their 3′ regions. Both SMN1 and SMN2 genes are able to be transcript; however, the SMN1 gene encodes stable and functional protein while the SMN2 gene encodes unstable protein for most of the time due to the lack of exon 7 of mRNA. As a result, clinical symptoms occur if there is a lack of SMN1 gene due to the deletion or replacement between SMN1 and SMN2 genes, and the expression level of SMN2 gene depends on the severity of the clinical symptoms.

Nowadays, the most commonly adopted method for detecting SMA is PCRFLP (Polymerase Chain Reaction Restriction enzyme Fragment Length Polymorphism). The gene fragment amplified by PCR contains SMN1 and SMN2, but only the SMN2 gene contains the nucleotide recognized and cut by restriction enzyme, not the SMN1 gene. Therefore, the gene fragment can be processed with restriction enzyme, and SMN1 and SMN2 genes can be detected by electrophoresis, but this method requires a longer reacting time.

Another method for detecting SMA is to sequence the nucleotide of the SMN gene, and then compare the differences between the nucleotides respectively. Despite the fact that this technique for sequencing can be conducted automatically, the costs of detecting equipment and materials are high, and the results need to be analyzed by well-trained technicians. Therefore, quantitative detection is not reasonable due to the longer time required and the high demands on labor force and costs.

The SMA patients and their families suffer from the high medical expenditure, and the related medical care is also a burden of the social resources. Most importantly, traditional detecting methods can only confirm the SMA after patients have fallen ill, and no detection method can screen the SMA carriers beforehand. Therefore, it is desirable to provide a prompt, accurate and economical method to detect the difference among the SMA genes, or provide genetic counseling or carrier screening in order to prevent this rare disease from occurring.

### SUMMARY OF THE INVENTION

The object of the present invention is to provide a detecting method for distinguishing the genes related to Spinal muscular atrophy. By using Denaturing High Performance Liquid Chromatography (DHPLC), developed by the research team of Professor Peter Oefner at Stanford University, USA, a minor mutation of even a single nucleotide can be detected automatically to assure and recognize the change of a single nucleotide. The principle of this technique is to heat up the PCR product to loosen the double helix DNA so that the distinction could be made from the mis-pairing of base pairs to the normal pairing. After obtaining the retention time via HPLC, the result can be gathered by UV detection.

To achieve the object, the steps of the present invention include: a) providing a genomic DNA; (b) amplifying the genomic DNA with a pair of primers to obtain amplified products; and (c) injecting the amplified products into DHPLC (Denaturing High Performance Liquid Chromatography).

The first goal in the method of the present invention is to amplify the survival motor neuron (SMN) gene which is the related gene fragment of Spinal muscular atrophy from genome. To successfully amplify the target gene, the pair of primers in step (b) preferably includes one forward primer, SEQ. ID NO. 1, and a reverse primer, which is SEQ. ID NO. 2, or any pair of primers that can successfully amplify products containing survival motor neuron gene. In addition, the amplifying reaction in step (b) is preferably polymerase chain reaction.

The method of the present invention further comprises a step (d) after the step (c), comparing the resulting pattern from DHPLC of step (c) to a standard control pattern of the SMN gene. Since the SMN1 and SMN2 genes with slight base difference can be identified by DHPLC, the standard control sample is based on the analysis of SMN1 and SMN2 by DHPLC and the differences between the retention times.

By using the method of amplifying the nucleotide with the SMN gene fragment specifically and conducting analysis via DHPLC, SMN1 and SMN2 genes with a tiny difference between them can be successfully distinguished. Furthermore, the existence of SMN1 and SMN2, or the ratio of these two genes is the determining factor for a non-SMA body. Therefore, the method of the present invention can detect not only the patients, but also the carriers of Spinal muscular atrophy.

Other objects, advantages, and novel features of the invention will become more apparent from the following detailed description when taken in conjunction with the accompanying drawings.

### BRIEF DESCRIPTION OF THE DRAWINGS

FIG. 1 shows the illustrations from DHPLC, wherein (a) represents an individual with an SMN2 gene only, and (b) represents an individual with an SMN1 gene only;

FIG. 2 shows a Chromatography and sequence analysis of a)an individual with equal dosage of SMN1/SMN2 genes, b)an individual with the SMN2 gene only, c)a construct with the SMN2 gene only, d)an individual with the SMN1 gene only, e)a construct with the SMN1 gene only;

FIG. 3 shows the illustrations from DHPLC of a)an individual with an SMN1/SMN2 gene ratio of one, b)an individual with a gene ratio of SMN1:SMN2=1:2, c)an individual with a gene ratio of SMN1:SMN2=1:3, d)an individual with a gene ratio of SMN1:SMN2=2:1, and e)an individual with a gene ratio of SMN1:SMN2=3:1;

FIG. 4 is a pedigree and DHPLC results of one core family. In this family, two sons had the SMN2 gene only; they are indicated as patients with SMA, and both father and mother were revealed to be carriers of SMA with an SMN1/SMN2 gene ratio of 1:3; and

FIG. 5 is a pedigree and DHPLC results for one core family. In this family, two sons had the SMN2 gene only and were shown to be patients with SMA; one daughter had a SMN1/SMN2 ratio of one which was classified as a normal variation; their father, mother, and the other daughter had a gene ratio of SMN1:SMN2=1:3 and were considered to be carriers.

### DETAILED DESCRIPTION OF THE PREFERRED EMBODIMENT

### EXAMPLE 1

Genomic DNA was collected from peripheral whole blood with a Puregene DNA Isolation Kit (Gentra Systems, Inc., Minneapolis, Minn., USA), according to the manufacturer's instructions.

### EXAMPLE 2

### Polymerase Chain Reaction

Polymerase chain reaction is performed to amplify SMN gene fragments in the genomic DNA to provide the sufficient DNA quantity for further detection.

Two almost identical copies of the SMN gene, telomeric SMN (SMN1) and centromeric SMN (SMN2), have been identified. These two SMN genes are highly homologous and differ in only five nucleotide exchanges within their 3′ regions. These variations do not alter the encoded amino acids. These nucleotide differences, located in exons 7 and 8, allow the SMN1 gene to be distinguished from the SMN2 gene. It has been reported that more than 95% of SMA patients were homozygous for deletion of the SMN1 gene. Moreover, small deletions or point mutations have been found in patients in whom SMN1 was present.

The SMN2 gene cannot compensate for the SMN1 deletion because, transiently, a single-nucleotide difference in exon 7 causes exon skipping. Therefore, detection of the absence of SMN1 can be a useful tool for the diagnosis of SMA. To detect the SMN1/SMN2 ratio, the intronic primers spanning exons 7 and 8 were used, where the sequence of SMN forward primer is 5′-TGTCTTGTGAAACAAAATGCTT-3′ as SEQ. ID NO. 1, and the reverse primer is 5′-AAAAGTCTGCTGGTCTGCCTA-3′ as SEQ. ID NO. 2.

PCR for the provided DNA fragments was performed in a total volume of 25 μL containing 100 ng of genomic DNA, 0.12 μM of each primer, 100 μM dNTPs, 0.5 unit of AmpliTaq Gold™ enzyme (PE Applied Biosystems, Foster City, Calif., USA), and 2.5 μL of GeneAmp 10× buffer II (10 mM Tris-HCl, pH=8.3, 50 mM KCl), in 2 mM MgCl₂, as provided by the manufacturer. Amplification was performed in a multiblock system (MBS) thermocycler (ThermoHybaid, Ashford, UK). PCR amplification was performed with an initial denaturation step at 95° C. for ten minutes, followed by 35 cycles consisting of denaturation at 94° C. for 30 seconds, annealing at 53° C. for 45 seconds, extension at 72° C. for 45 seconds, and then a final extension step at 72° C. for ten minutes.

### EXAMPLE 3

### DHPLC Analysis

The DHPLC system used in this study is a Transgenomic Wave Nucleic Acid Fragment Analysis System (Transgenomic Inc., San Jose, Calif.). DHPLC was carried out on automated HPLC instrument equipped with a DNASep column (Transgenomic Inc., San Jose, Calif.). The DNASep column contains proprietary 2-mm nonporous alkylated poly (styrenedivinylbenzene) particles. The DNA molecules eluted from the column are detected by scanning with a UV detector at 260 nm. DHPLC-grade acetonitrile (9017-03, J. T. Baker, Phillipsburg, N.J., USA) and triethylammonium acetate (TEAA, Transgenomic™, Crewe, UK) constituted the mobile phase. The mobile phases consisted of 0.1 M TEAA with 500 μL of acetonitrile (eluent A) and 25% acetonitrile in 0.1 M TEAA (eluent B).

For heteroduplex and multiplex detection, crude PCR products obtained from example 2 were subjected to an additional 5-min 95° C. denaturing step followed by gradual reannealing from 95° C. to 25° C. over a period of 70 min. The start and end points of the gradient were adjusted according to the size of the PCR products by use of an algorithm provided by WAVEmaker™ system control software (Transgenomic Inc., San Jose, Calif.).

Twenty μL of PCR product was injected for analysis in each run. The samples were run under partially denaturing conditions according to the nature of each amplicon and provided by WAVEmaker™ system control software (Transgenomic Inc., San Jose, Calif.). The buffer B gradient increased 2% per minute for 4.5 minutes at a flow rate of 0.9 mL/min. Generally, the analysis took about 10 min for each injection.

### EXAMPLE 4

### Cloning and Sequencing of PCR-Generated DNA Fragments

To generate DNA fragments for use as positive controls in PCR reactions and to facilitate DNA sequencing, the PCR fragment of the SMN1 gene and SMN2 gene were subcloned into pGEM®-T Easy Vector (Promega Corporation, Madison, Wis.), followed by digestion according to the manufacturer's instructions.

For cloning, 5 μL of the PCR fragment were mixed with pGEM®-T Easy Vector in a final volume of 10 μL and ligated at 4° C. overnight. Then 5 μL of the recombinant plasmid was used for transformation into E. coli, which was then cultured overnight on selective agar plates containing 20 μL of 50 g/L of ampicillin. The plates were incubated at 37° C. overnight. White colonies were randomly chosen and were routinely cultured at 37° C. overnight on LB broth containing ampicillin. Recombinant plasmid DNA was extracted and purified by a Mini-M™ Plasmid DNA Extraction System (Viogene, Sunnyvale, Calif.).

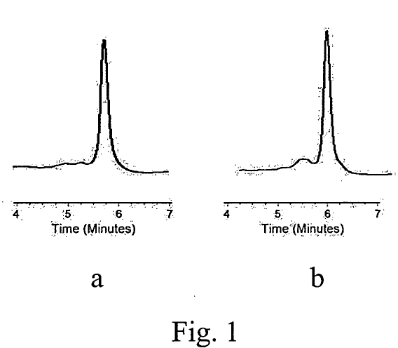

Extracted plasmid DNA was then subjected to DHPLC analysis, and the results of SMN1 and SMN2 are shown in FIG. 1 as standard patterns. FIG. 1a is the pattern of SMN1 with retention time less than 6 minutes, and 1b is SMN2 with retention time at about 6 minutes.

### EXAMPLE 5

### Direct Sequencing

Amplicons were purified by solid-phase extraction and bidirectionally sequenced with the PE Biosystems Taq DyeDeoxy terminator cycle sequencing kit (PE Biosystems) according to the manufacturer's instructions. Sequencing reactions were separated on a PE Biosystems 373A/3100 sequencer. The resulting patterns were compared to the patterns from DHPLC of example 3.

### EXAMPLE 6

### Quantitative Real-Time PCR of SMN1 and SMN2 Copy Numbers

TaqMan™ technology was used for determination of SMN1 and SMN2 gene dosages. Quantification was performed with an ABI Prism 7000 sequence detection system and 96-well MicroAmp optical plates (Applied Biosystems). The SMN genes were amplified by use of the forward primer 5′-AATGCTTTTTAACATCCATATAAAGCT-3′ as SEQ. ID NO. 3, and the reverse primer 5′-CCTTAATTTAAGGAATGTGAG CACC-3′ as SEQ. ID NO. 4. The MGB (minor groove binder) probes (Applied Biosystems) were designed to distinguish between the SMN1 and SMN2 genes in exon 7 at position 6. The two specific hybridization probes were labeled with 5′-FAM as a fluorescent dye (SMN1-Ex7: 5′-CAGGGTTT CAGACAAA-3′ is coded SEQ. ID NO. 5 and SMN2-Ex7-anti: 5′-TGATTTTGTCTAA AACCC-3′ is coded SEQ. ID NO. 6).

PCR was performed in a total volume of 25 μL containing 50 ng of genomic DNA, 0.3 μM of each primer, 13 μL Platinum® qPCR Supermix-UDG (Invitrogen, Karlsruhe, Germany), 0.5 mM ROX as a passive reference (Invitrogen), 2 mM MgCl₂, and 100 nmol of each MGB probe. The 96-well plate contained 125 ng, 25 ng, and 5 ng standard DNA, respectively. Each test sample and each amount of standard DNA were run in duplicate. All reactions of the same run were prepared from the same master mix.

Reactions for the SMN1 or SMN2 test loci and the Factor VIII gene reference locus were prepared and run in parallel. The PCR conditions were one cycle at 50° C. for 2 min, one cycle at 95° C. for 10 min, followed by 40 cycles of 95° C. for 15 s, 60° C. for 1 min. The analysis was performed with ABI 37000SDS software (Applied Biosystems).

### EXAMPLE 7

In the result of DHPLC analysis, peaks appearing in different retention times indicate various forms of DNA were detected. The binding force between DNA molecules is changed in different oven temperatures of DHPLC, and the SMN1/SMN2 peaks were identified unambiguously at different oven temperatures, thus to recognize single base difference between SMN1 and SMN2.

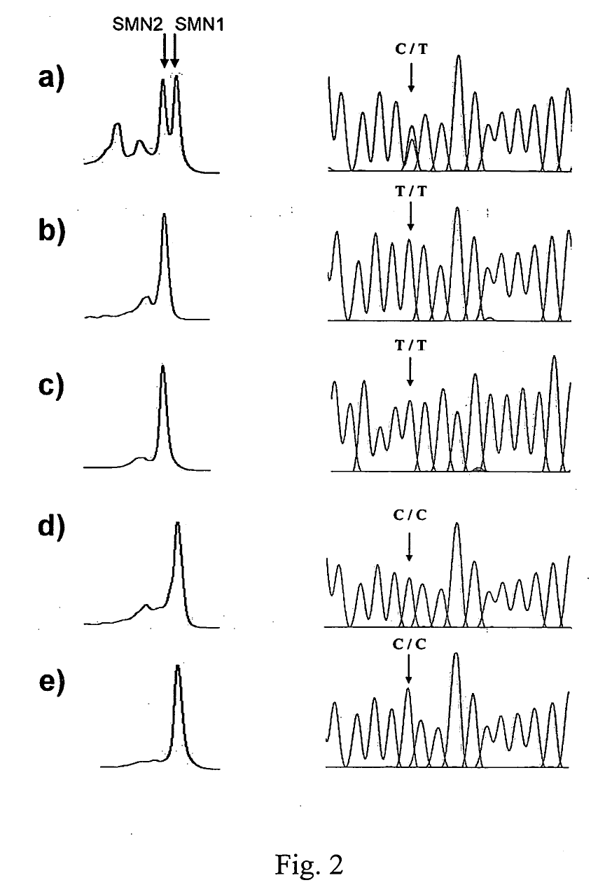

FIG. 2 shows the pattern of DHPLC and the sequencing result of example 5. Both SMN1/SMN2 genes are found in FIG. 2a) and a peak representing single nucleotide mutation(cytosine-thymine mutant) can be identified in the corresponding sequencing result. That is, a heteroduplex DNA sample. FIG. 2b) (from plasmid SMN2) and FIG. 2c) (from human DNA) both show patterns indicating a homoduplex DNA with thymine. These revealed that both two DNA samples contained gene fragments of SMN2. Also, the patterns of FIG. 2d) (from plasmid SMN1) and FIG. 2e) (from human DNA) both show a homoduplex DNA sample with cytosine, and the DNA samples are SMN1 genes-containing. Example 8 By DHPLC analysis, the peaks of SMN1/SMN2 can be identified unambiguously at different oven temperatures. It was proved that the SMN1/SMN2 peak ratio detected by DHPLC at 52.5° C. oven temperature was compatible with gene dosages determined by quantitative real-time PCR analysis. To test the validity and reproducibility of the detection system for gene dosage determination for the SMN1/SMN2 genes, every sample was repeatedly analyzed at least three times and all demonstrated the reproducible results.

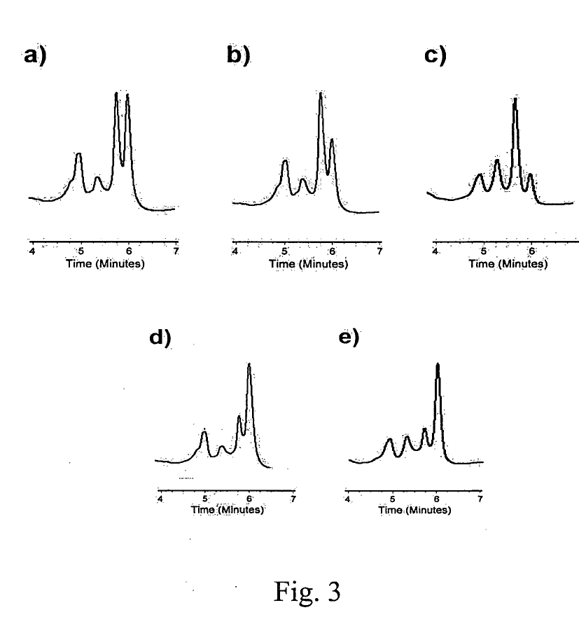

As shown in FIG. 3, different dosages of SMN1 and SMN2 can be distinguished clearly in FIG. 3a-e. FIG. 3a shows ratios of the dosages of SMN1 and SMN2 are the same. SMN1:SMN2=1:2 on FIG. 3b, SMN1:SMN2=1:3 on FIG. 3c, SMN1:SMN2=2:1 on FIG. 3d, and SMN1:SMN2=3:1 on FIG. 3e.

### EXAMPLE 9

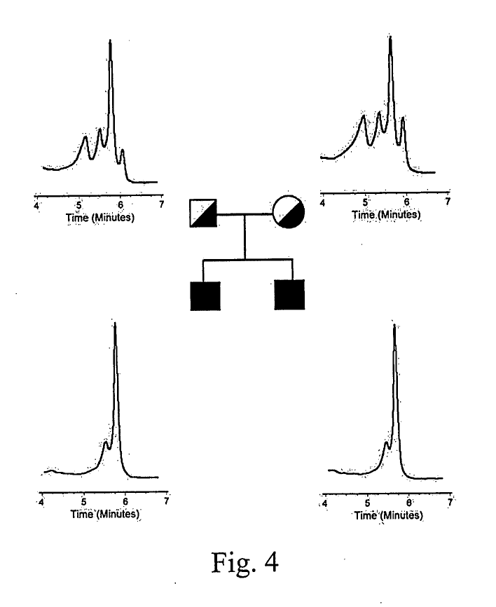

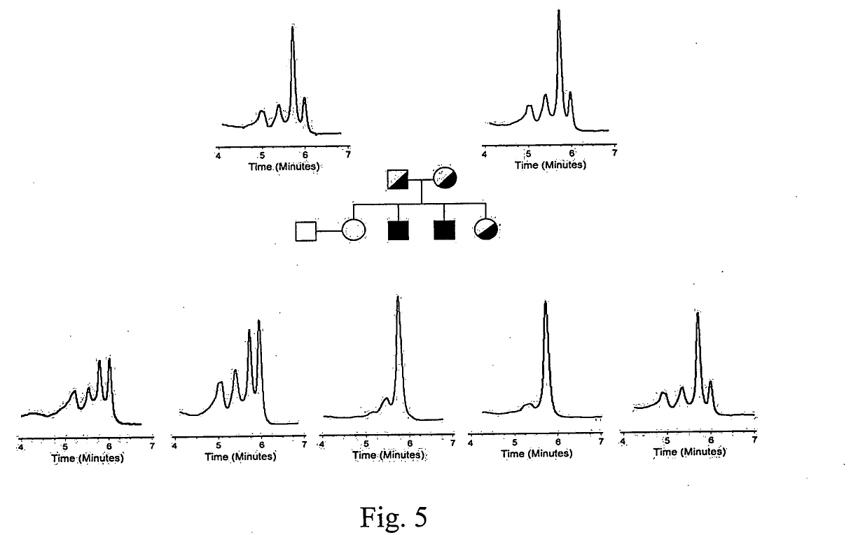

Analysis on the pedigrees of two families with SMA is conducted by the screening method of the present invention, and the results are shown in FIGS. 4 and 5. On FIG. 4, both the father ( [symbol] ) and the mother ( [symbol] ) are carriers, and the two sons are recognized as SMA patients (▪). The analysis from DHPLC shows that the ratio of the SMN genes for the father is SMN1:SMN2=1:3, SMN1:SMN2=1:3 for the mother, and SMN 1:SMN2=0:4 which results in the morbidity of SMA due to the lack of the SMN1 gene.

FIG. 5 reveals the pedigree of the other family in which the father ( [symbol] ) and the mother ( [symbol] ) are both carriers. Among the four children they have, two sons (▪) who are SMA patients while one daughter is a non-carrier (◯) and the other is identified as a carrier ( [symbol] ). In addition, a healthy man who is married to the healthy daughter is also a non-carrier. The analysis from DHPLC shows that the ratio of SMN genes for the father is SMN1:SMN2=1:3, SMN1:SMN2=1:3 for the mother, SMN1:SMN2=0:4 for the two sons, SMN1:SMN2=1:3 for the carrier daughter, SMN1:SMN2=2:2 for the non-carrier daughter, and SMN1:SMN2=2:2 for the healthy man married to the non-carrier daughter.

In this invention, it is successfully demonstrated to apply DHPLC, which is originally used on detecting the mutation of a single nucleotide, to the screening test for the SMA patients and carriers. On the patterns of the results, the result of a single peak represents the existence of homozygote, and the change of the retention time, the change of the peaks, shows the existence of heterozygote. The present invention can distinguish the existences of homozygote and heterozygote, and it compensates for the drawbacks of direct sequencing such as its longer time consumption and high costs in order to detect the patients and carriers efficiently, economically, and accurately within a short period of time. Compared with the ongoing method, the method of the present invention not only contains the characteristics of being fast, more accurate and of higher sensitivity when detecting the patients, as for the carriers, it also provides a method for further screening with speed and accuracy.

Although the present invention has been explained in relation to its preferred embodiment, it is to be understood that many other possible modifications and variations can be made without departing from the scope of the invention as hereinafter claimed.

What is claimed is:

1. A method for identifying SMA(spinal muscular atrophy)-affected patients comprising the steps of:
(a) providing a genomic DNA;
(b) amplifying the genomic DNA with a pair of primers to obtain amplified products; and
(c) injecting the amplified products into DHPLC (Denaturing High Performance Liquid Chromatography).

2. The method as claimed in claim 1, wherein said primers in step (b) comprise a forward primer that consists the nucleotide sequence set forth in SEQ. ID NO. 1.

3. The method as claimed in claim 1, wherein said primers in step (b) comprise a reverse primer that consists the nucleotide sequence set forth in SEQ. ID NO. 2.

4. The method as claimed in claim 1, wherein said amplifying in step (b) is performed by polymerase chain reaction.

5. The method as claimed in claim 1, wherein certain gene fragments correlating to spinal muscular atrophy are contained in said amplified products of step (c).

6. The method as claimed in claim 5, wherein said certain fragments correlating to spinal muscular atrophy are survival motor neuron (SMN) gene.

7. The method as claimed in claim 1, further comprising a step (d) after step (c), comparing the resulting illustrations from DHPLC of step (c) to a standard illustration of SMN gene.

8. The method as claimed in claim 1, which is enabling for spinal muscular atrophy carrier screening.
